(19) United States
(12) Patent Application Publication
(10) Pub. No.: US 2015/0079534 A1
(43) Pub. Date: Mar. 19, 2015

(54) APPARATUS FOR IMAGING DENTAL ARCH IN ORAL CAVITY

(71) Applicants: Hironobu Tsuji, Tokyo (JP); Kazufumi Suzuki, Tokyo (JP)

(72) Inventors: Hironobu Tsuji, Tokyo (JP); Kazufumi Suzuki, Tokyo (JP)

(73) Assignee: Media Co., Ltd., Tokyo (JP)

(21) Appl. No.: 14/377,516

(22) PCT Filed: Jul. 26, 2012
(86) PCT No.: PCT/JP2012/068986

§ 371 Date: Nov. 22, 2014

(30) Foreign Application Priority Data
Feb. 15, 2012 (JP) 2012-030193

(51) Int. Cl. A61C 9/00; A61B 1/247

(52) U.S. Cl. CPC A61C 9/006; A61B 1/247
USPC 433/29

(57) ABSTRACT

There is provided an intraoral teeth image taking apparatus capable of simultaneously taking from a plurality of directions a group of high-precision images for calculation of high-precision three-dimensional data required, for example, when artificial teeth are made. The intraoral teeth image taking apparatus includes an image taking tray part 101 having a plurality of image taking devices 121 capable of simultaneously taking a plurality of images of a row of teeth in an oral cavity from a plurality of directions, and a plurality of projection devices 151 capable of projecting a plurality of patterns. The image taking tray part is formed in a U-shape in conformity with the shape of the row of teeth and the cross-sectional shape of the tray is concave such as to be capable of being put on a distal end portion of the row of teeth.

### TECHNICAL FIELD

The present invention relates to an intraoral teeth image taking apparatus used to take images of a row of teeth in an oral cavity and, more particularly, to an intraoral teeth image taking apparatus capable of simultaneously taking from a plurality of directions a group of high-precision images for calculation of high-precision three-dimensional data required, for example, when artificial teeth are made. The taken images can also be used for preparation of mosaic images for diagnosis.

### BACKGROUND ART

A method of calculating three-dimensional coordinates of points on an object from planar images (two-dimensional images) of the object taken with a plurality of image taking devices is known. This is called “three-dimensional reconstruction from planar images”. An apparatus for taking images from a row of teeth in an oral cavity with a plurality of image taking devices has been disclosed. This apparatus performs three-dimensional reconstruction of a row of teeth by calculation from a plurality of planar images taken (see, for example, Patent Literature 1).

It is necessary to take images with a plurality of image taking devices (cameras) maintained in the same state as at the time of calibration in order to obtain planar images to be used for three-dimensional reconstruction. That is, when three-dimensional reconstruction is performed by using a plurality of planar images taken in a state different from the state at the time of calibration, it is difficult to achieve a sufficiently high degree of accuracy. A state where conditions at the time of calibration are held will hereinafter be referred to as “calibrated condition holding state”. For example, if the positional relationship between the plurality of image taking devices and the base line lengths (the distances between the cameras) or the like are changed from their states at the time of calibration, the calibrated condition holding state is collapsed.

When images are taken while the image taking devices are moved, the images are taken in a state where the positional relationship and the base line lengths are changed and the calibrated condition holding state is collapsed, unless the images are simultaneously taken with the plurality of image taking devices. In the method disclosed in Patent Literature 1, no particular measures are taken to cope with this. When this method is used, the base line lengths are changed because the image taking devices or the image taking operator is slightly moved at all times, and it is difficult to hold the calibrated condition holding state. When three-dimensional reconstruction from planar images taken under such a condition is performed, it is difficult to achieve a sufficiently high degree of accuracy.

### CITATION LIST

### Patent Literature

Patent Literature 1: Japanese Patent Laid-Open No. 2010-069301

### SUMMARY OF INVENTION

### Technical Problem

The present invention has been achieved in consideration of the above-described problem, and an object of the present invention is to provide an intraoral teeth image taking apparatus capable of simultaneously taking from a plurality of directions a group of high-precision images for calculation of high-precision three-dimensional data required, for example, when artificial teeth are made.

### Solution to Problem

To achieve the above-described object, according to the present invention, an intraoral teeth image taking apparatus described below is provided.

A first intraoral teeth image taking apparatus includes an image taking tray part for taking images of a tooth or a row of teeth in an oral cavity, and a grip part. In this apparatus, a plurality of image taking devices capable of simultaneously taking images from a plurality of directions and a plurality of projection devices capable of projecting a plurality of radiation patterns are provided on a surface of the tray part.

According to a second intraoral teeth image taking apparatus, in the first intraoral teeth image taking apparatus, the image taking tray part is formed in a U-shape in conformity with the shape of a row of teeth and the cross-sectional shape of the tray is concave such as to be capable of being put on a distal end portion of the row of teeth.

According to a third intraoral teeth image taking apparatus, in the first or second intraoral teeth image taking apparatus, each image taking device is provided with an electric shutter mechanism for limiting the picture taking time and a sync image taking mechanism electrically connected to the electric shutter mechanisms and capable of causing the electric shutter mechanisms to simultaneously perform image taking operations.

According to a fourth intraoral teeth image taking apparatus, in any of the first or second intraoral teeth image taking apparatus, each image taking device is provided with a transmitter capable of transmitting a signal transmittable wirelessly.

According to a fifth intraoral teeth image taking apparatus, in any of the first or second intraoral teeth image taking apparatus, each projection device is capable of changing the radiation pattern.

According to a sixth intraoral teeth image taking apparatus, in any of the first or second intraoral teeth image taking apparatus, the image taking tray part is divided into two arm portions swingably connected by a hinge and is provided with adjustment means for enabling adjusting the angle between the arm portions.

### Advantageous Effects of Invention

According to the invention of the first intraoral teeth image taking apparatus, simultaneously taking from a plurality of directions a group of high-precision planar images for calculation of high-precision three-dimensional data required, for example, when artificial teeth are made, is enabled and the effect of enabling easy image taking in the calibrated condition holding state can be obtained. The effect of enabling use for preparing mosaic images for diagnosis is also obtained. According to the invention of the second intraoral teeth image taking apparatus, the effect of enabling taking images of a row of teeth in an oral cavity from suitable positions and directions can be obtained. According to the invention of the third intraoral teeth image taking apparatus, the effect of taking accurate images of a row of teeth under projection of a suitable pattern of light can be obtained.

### BRIEF DESCRIPTION OF DRAWINGS

FIG. 1 is a diagram schematically showing the configuration of an intraoral teeth image taking apparatus according to the first embodiment of the present invention as seen from the front side of an image taking tray part (the image taking object teeth side).

FIG. 2 shows the structure of the image taking tray part in the intraoral teeth image taking apparatus shown in FIG. 1; FIG. 2(a) is a sectional view taken along line A-A in FIG. 1; and FIG. 2(b) is an enlarged view of a portion B of FIG. 2(a).

FIG. 3 is a plan view schematically showing a modified example of the intraoral teeth image taking apparatus according to the embodiment of the present invention (a view of an arrangement enabling adjustment of the angle between arm portions of the image taking tray part).

### DESCRIPTION OF EMBODIMENT

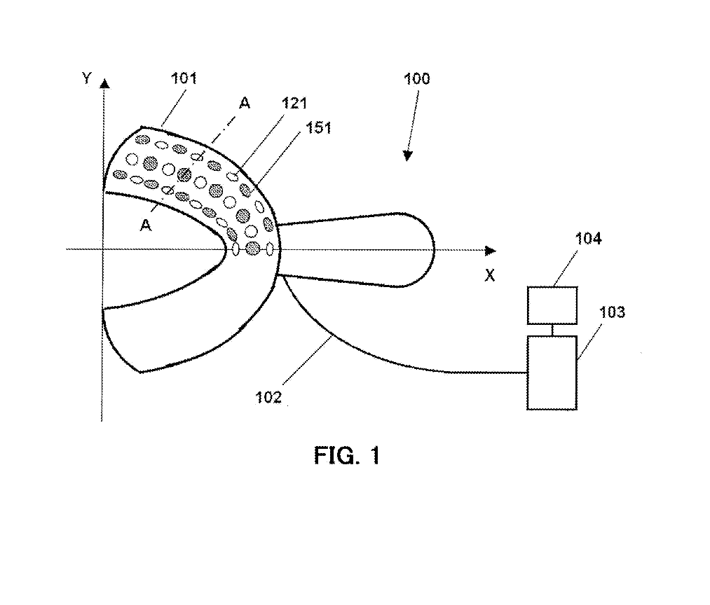

An embodiment of an intraoral teeth image taking apparatus according to the present invention will be described concretely with reference to FIGS. 1 to 3. FIG. 1 is a diagram schematically showing the construction of an intraoral teeth image taking apparatus according to a first embodiment of the present invention as seen from the front side of an image taking tray part. As shown in FIG. 1, in the construction of the intraoral teeth image taking apparatus 100 according to the first embodiment of the present invention schematically shown, there is provided an image taking tray part 101 that has a plurality of image taking devices (cameras) 121 capable of simultaneously taking images of a row of teeth in an oral cavity from a plurality of directions and a plurality of projection devices 151 capable of projecting a plurality of radiation patterns.

As shown in FIG. 1, the intraoral teeth image taking apparatus 100 according to the present embodiment is further provided with a cable 102, a sync image taking mechanism (not shown), a computer 103 and a monitor 104.

The image taking devices 121 are capable of simultaneously taking images of a row of teeth in an oral cavity from a plurality of directions, as described above. In addition, the image taking devices 121 are also capable of taking images of the row of teeth in the oral cavity from a plurality of directions with passage of time. It is assumed that “simultaneous” in simultaneous image taking in the present invention is broadly interpreted as also denoting a case where image taking is performed at consecutive times while maintaining the same calibrated condition holding state.

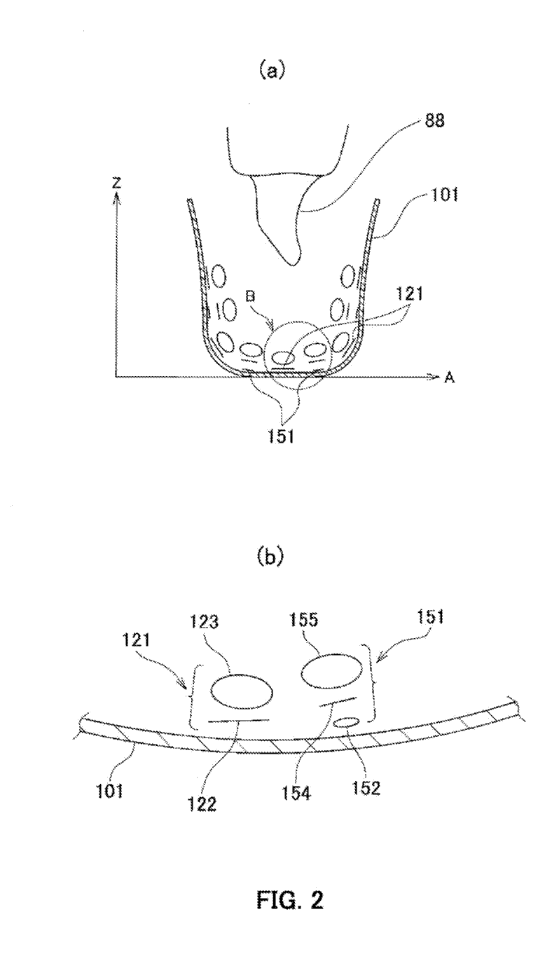

X, Y and Z in FIG. 1 and FIGS. 2 and 3 referred to below respectively represent the coordinate axes of a three-dimensional coordinate system. The abscissa A in FIG. 2 is a representation of line A-A in FIG. 1 as coordinate axis A.

FIG. 1 is a view of the intraoral teeth image taking apparatus 100 according to the present embodiment as seen from the front side (the upper side as viewed in FIG. 2) of the image taking tray part 101 having the shape of a dental impression tray. As shown in FIG. 1, the plurality of image taking devices 121 and the plurality of projection devices 151 are disposed so as to be capable of taking images of an image taking object at least from two directions. The projection devices 151 apply patterns of light for detecting corresponding points of a plurality of images. Data of images taken by the image taking devices 121 is sent to the computer 103 through the cable 102 for display on the monitor 104. A grip part (with no reference numeral) is shown on the right-hand side of the image taking tray part 101 in FIG. 1.

FIG. 2(a) is a sectional view taken along line A-A in FIG. 1, showing the structure of the image taking tray part 101 in the intraoral teeth image taking apparatus 100 shown in FIG. 1. As shown in FIG. 2(a), the image taking devices 121 and the projection devices 151 are alternately disposed on inner bottom portions of the image taking tray part 101. FIG. 2(b) is an enlarged view of a portion B of FIG. 2(a). Each image taking device 121 is constituted by an image pickup element 122 and a lens 123. Each projection device 151 is constituted by a light source 152, a pattern generator 154 and a projection lens 155.

The image pickup element 122 may be, for example, a CCD or CMOS configured in planar form. The lens 123 may be a single lens or a combination of a plurality of lenses made of glass or a plastic and formed so as to have a spherical or aspherical surface. The light source 152 may be, for example, a light emitting diode (LED) or an incandescent lamp. The pattern generator 154 may be, for example, a film or a liquid crystal. The projection lens 155 may be a single lens or a combination of a plurality of lenses made of glass or a plastic and formed so as to have a spherical or aspherical surface.

Each image taking device 121 has an electric shutter mechanism for limiting the image taking time, not shown. The electric shutter mechanism changes (turns on/off) an electrical signal with an electronic circuit and has no mechanical structure. The electric shutter mechanisms provided in the plurality of image taking devices 121 are electrically connected to each other and are arranged to simultaneously perform image taking operations with the sync image taking mechanism not shown. The sync image taking mechanism may be arranged to cause the electric shutter mechanisms to perform the image taking operations with passage of time.

Each electric shutter mechanism is operated by an electric signal. The image taking device 121 accumulates electric charge according to the lightness of the image taking object while the electric signal is being applied. It is preferable to take images by opening the shutter of the electric shutter mechanism for a sufficiently short time, e.g., 0.1 to 10.0 ms, such that the image is not blurred even if the image taking tray part 101 is shaken.

With the above-described arrangement, images from a plurality of directions including directions from two side surfaces of teeth are taken in synchronization with each other by means of the plurality of image taking devices 121. The plurality of images taken are sent to the computer 103 to be processed.

Calibration for three-dimensional reconstruction with the plurality of image taking devices 121 can be performed, for example, by putting the image taking tray part 101 on a three-dimensionally measured teeth model (not shown) or a three-dimensionally measured standard object (not shown) as in the case of ordinary image taking and by thereafter performing image taking. Calibration may be performed at any time before or after image taking. Calibration may be omitted in the case of preparation of mosaic images.

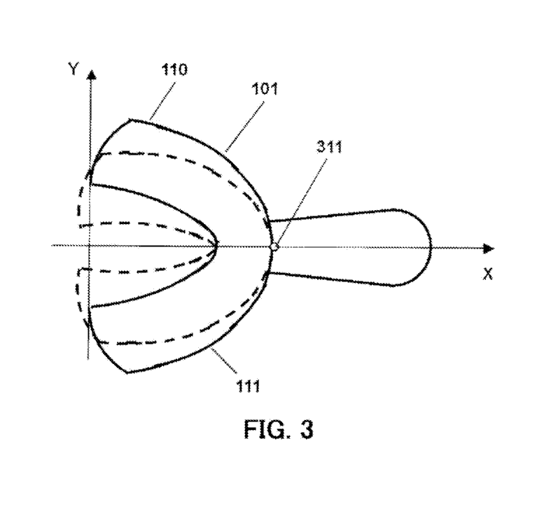

FIG. 3 is a plan view schematically showing a modified example (arranged so that the angle between arms of the image taking tray part 101 is adjustable) of the intraoral teeth image taking apparatus according to the embodiment of the present invention. The configuration of a row of teeth may vary depending on the jaw size as between an adult person and a young child. For the purpose of suitably maintaining the distances between the row of teeth and the lenses in such a case, two arms 110 and 111 of the image taking tray part 101 may be made swingable on a hinge 311 as shown in FIG. 3, and adjustment means may be provided to enable adjustment of the arms. A potion indicated by a broken line in FIG. 3 represents a state of the arms after the arms are adjusted by being inwardly turned. For example, as adjustment means (not shown), nuts reverse threaded relative to each other may be fixed on the two arms 110 and 111 and a threaded rod may be screw-fitted therein to enable adjustment.

In the first embodiment shown in FIG. 1, images taken with the image taking devices 121 are sent from the image taking devices 121 to the computer 103 through the cable 102 to be displayed on the monitor 104. In another modified example of the apparatus, a transmitter that transmits a wireless signal such as an infrared signal or an electromagnetic wave signal may be connected to the plurality of image taking devices 121 to transmit the wireless signal to the computer 103. This arrangement enables removal of the cable 102 shown in FIG. 1 and simplification of the configuration.

Another modified example is conceivable in which the projection device 151 shown in FIG. 2 is arranged so that the on/off positions in the liquid crystal (pattern generator) 154 for generating patterns can be changed to enable the image taking device 121 to take a plurality of different planar images. The accuracy with which corresponding points are detected can be improved in this way. Calculation for three-dimensional reconstruction requires detection of corresponding points in a plurality of planar images. In general, the accuracy is improved if the amount of data is larger. More specifically, detection of corresponding points by taking images is repeated a certain number of times while changing the projection pattern, and all the plurality of corresponding points thereby detected are used or the medians, averages or the like of the plurality of corresponding points are used, thereby improving the accuracy. An improvement in accuracy can also be achieved by slightly moving the image taking tray part 101 each time images are taken.

### REFERENCE SIGNS LIST

88 Row of teeth in oral cavity

100 Intraoral teeth image taking apparatus

101 Image taking tray part

102 Cable

103 Computer

104 Monitor

110 Arm

111 Arm

121 Image taking device

122 Image pickup element

123 Lens

151 Projection device

152 Light source

154 Pattern generator

155 Projection lens

311 Hinge

1. An intraoral teeth image taking apparatus comprising an image taking tray part for taking images of a tooth or a row of teeth in an oral cavity, and a grip part, wherein a plurality of image taking devices capable of simultaneously taking images from a plurality of directions and projection devices capable of projecting a plurality of radiation patterns are provided on a surface of the tray part, and wherein each projection device is arranged so as to be able to generate a plurality of patterns by changing a projection device which is successively turned on and off, and to thereby enable taking a plurality of planar images.

2. The intraoral teeth image taking apparatus according to claim 1, wherein the image taking tray part is formed in a U-shape in conformity with the shape of a row of teeth and the cross-sectional shape of the tray is concave so as to be capable of being put on a distal end portion of the row of teeth.

3. The intraoral teeth image taking apparatus according to claim 1 or 2, wherein each image taking device is provided with an electric shutter mechanism for limiting the picture taking time and a sync image taking mechanism electrically connected to the electric shutter mechanisms and capable of causing the electric shutter mechanisms to simultaneously perform image taking operations.

4. The intraoral teeth image taking apparatus according to claim 1 or 2, wherein each image taking device is provided with a transmitter capable of transmitting a signal transmittable wirelessly.

5. (canceled)

6. The intraoral teeth image taking apparatus according to claim 1 or 2, wherein the image taking tray part is divided into two arm portions swingably connected by a hinge and is provided with adjustment means for adjusting the angle between the arm portions.
